SEQUENCE LISTING (1 sequence)

SEQ ID NO: 1 - DNA, 23 bp, unidentified
  source 1..23: mol_type = other DNA
aagcagtggtatcaacgcagagt

(19) United States
(12) Patent Application Publication
(10) Pub. No.: US 2025/0230494 A1
(43) Pub. Date: Jul. 17, 2025

(54) METHODS OF SINGLE CELL RNA-SEQUENCING

(71) Applicant: YEDA RESEARCH AND DEVELOPMENT CO. LTD., Rehovot (IL)

(72) Inventors: Moshe Biton, Rehovot (IL); Carmel Sochen, Rehovot (IL)

(21) Appl. No.: 18/854,052

(22) PCT Filed: Apr. 4, 2023
(86) PCT No.: PCT/IL2023/050362

§ 371 Date: Oct. 3, 2024

(30) Foreign Application Priority Data
Apr. 14, 2022 (IL) 292281

(51) Int. Cl. C12Q 1/6869; C12Q 1/6806; C12Q 1/6853; C12Q 1/686; C40B 50/08

(52) U.S. Cl. CPC C12Q 1/6869; C12Q 1/6806; C12Q 1/6853; C12Q 1/686; C40B 50/08

(57) ABSTRACT

The present invention provides methods and compositions for analysis multiplexed RNA transcriptomes at the single cell level.

### FIELD OF THE INVENTION

The present invention relates to methods and compositions for single cell RNA-sequencing and analysis. In particular, the present invention provides improved high-throughput, multiplexed and targeted methods for transcriptomic analysis at the single cell level.

### BACKGROUND OF THE INVENTION

RNA sequencing (RNA-seq) is a genomic analytical tool aimed at the detection and quantification of messenger RNA molecules, and is useful for studying the distinct cellular responses of individual constituents in a biological sample, particularly a complex entity such as a tissue or organ. RNA-seq can reveal valuable data regarding real-time gene expression and its level in response to a particular stimulus, and inter-tissue variations in gene expression profiles. Specific gene expression fluctuations can occur in response to environmental stimuli, as a function of different developmental stages, or in direct response to a pathophysiological situation. For practical reasons, the technique is usually conducted on samples comprising thousands to millions of cells, and requires a pooling step, which albeit yielding a vast amount of information, does not allow a detailed assessment of the fundamental biological unit, the cell or the individual nuclei that package the genome.

Single-cell RNA sequencing (scRNA-seq) technologies allow RNA-seq to be performed on single cells and thus can investigate RNA expression differences on a cell-by-cell basis. Hence, scRNA-seq enables statistical analyses that can yield more biological insights than traditional RNA-seq. For example, cell-to-cell variations are often observed within cancerous and embryonic cell samples. However, these variations cannot be detected by bulk RNA-seq (Yip, et al. Briefings in Bioinformatics, 20(4), 2019, 1583-1589).

The most commonly used scRNA-seq methods include the 10× Genomics Chromium, Smart-seq2 (SS2), Mars-seq and CEL-seq2, designed to answer different biological questions. There are several fundamental differences between the methods and each method has its advantages and drawbacks. For example, the amplification step, which in 10× and SS2 is done via PCR amplification, Mars-seq2 and CEL-seq2 utilize in vitro transcription (IVT). IVT results in an RNA product, which is sensitive to degradation, thus potentially leading to product loss during sample handling. In addition to amplification, Mars-seq uses [symbol] eal Illumina-based adapters required for RNA-sequenci [symbol] rocess is known to be less efficient than primer annealing processes, leading to product loss.

In terms of a platform, 10× Chromium is a microfluidics-based method. In microfluidics-based methods, all cells are loaded at the same time, with usually around 8,000 cells per channel, with up to 8 channels in the 10× chromium chip platform. Thus, microfluidics is a powerful platform since it allows the simultaneous sequencing of thousands of cells (up to 64000 cells in one go in the current version), and is easily performed. However, its main limitation is that sequenced cells need to be freshly isolated from the tissue or for frozen cells/tissues nuclei preparation is needed. Therefore, in cases of long experiments with several time points or human sample acquisition, all samples need to be collected at the same time, which is not always experimentally possible. The alternative is to sequence each time point or sample separately. However, this introduces batch effects, reducing the ability to analytically distinguish between sample variability caused by biological processes, compared with variability due to technical sample processing.

An alternative to microfluidics platforms is well-based sequencing methods such as Mars-seq, SS2 and CEL-seq2. Well-based sequencing is the collection of a single cell into each well of a 96 or 384-well plate. Cells are most commonly collected using fluorescent activated cell sorting (FACS). In this manner, collected cells can be stored in well plates for elongated time periods, thus allowing the accumulation of samples from different experiments, eventually preparing libraries from all experiments together, and thus reducing batch effects in the analysis. Well-based methods are extremely beneficial in the case of human sample collection, when samples are often obtained at different time points, yet they can still be prepared for sequencing together if multiplexing of plates is possible.

A disadvantage of well-based sequencing is the relatively reduced throughput ability compared with microfluidics-based methods (apart from 10× genomics, other worth mentioning methods are Drop-seq and inDrop). A single plate usually contains up to 384 cells, where each well is individually processed for library preparation, which is labor intensive, time consuming and usually expensive.

Multiplexing solves this issue, greatly increasing the throughput of well-based methods. With multiplexing it is possible to pool together hundreds or thousands of cells using cell-specific barcodes, thus making the throughput ability of plate-based methods comparable to that of microfluidics methods. Well-based multiplexing is acquired by sample pooling of all wells into a single well, processing all samples as an individual sample, thus reducing [symbol] costs. Pooling is possible thanks to cell barcode sequ [symbol] duced to the library structure at the first step of reverse transcription.

After a cell barcode sequence is annealed to the RNA and becomes part of the cDNA, all samples can be combined. The individual samples are demultiplexed during the computation analysis following sequencing as in 10× genomics. Mars-seq2 and CEL-seq2 both utilize pooling to improve their cell processing abilities, making them high-throughput methods.

WO 2018/222548 discloses methods for amplifying RNA using a combination of reverse transcription and multiple annealing and looping based amplification cycles. Primers are used such that the resulting amplicons include a first cell specific barcode sequence, a second cell specific barcode sequence and a unique molecular identifier barcode sequence.

WO 2020/180778 discloses methods for preparing a sequencing library that includes nucleic acids from a plurality of single cells. The methods include nuclear or cellular hashing which permits increased sample throughput and increased doublet detection at high collision rates.

US Patent application No. 2021/0047638 discloses methods for preparing a Next Generation Sequencing (NGS) library from an RNA Sample.

There is still an unmet need for improved, robust, and cost-effective methods for single-cell RNA sequencing and analysis.

### SUMMARY OF THE INVENTION

The present invention provides methods and compositions for single-cell RNA sequencing (scRNA-seq), the methods comprising reverse transcription, template switching, pooling, amplification, and tagmentation. The methods of the present invention further comprise a step of generating a complementary strand using gene-specific primers. The methods of the invention enable the enrichment, detection and quantification of rare sequences and/or of any desired genes of interest in parallel to whole transcriptomic analysis.

The methods and systems of the present invention are sensitive and accurate, and enable incorporation of cell barcodes for pooling libraries, thus allowing for processing different libraries together, reducing batch effects and increasing throughput.

It is now disclosed that even though a low volume of starting genetic material may be used, the quality of the sequencing data, and accordingly the genetic information that can be derived therefrom, is very high and enable sensitive and comprehensive mapping of the [symbol] s of the sequenced cells. Advantageously, the methods [symbol] ovide a comprehensive data about the whole transcriptome in parallel to the enrichment and focus on rare and/or desired genes of interest.

According to one aspect, the present invention provides a method for preparing a library of nucleic acids for sequence analysis of single-cell transcriptomes, comprising:
(i) providing a plurality of RNA populations of individual cells, wherein the RNA populations being separated;
(ii) reverse-transcribing the plurality of RNA populations using a plurality of reverse transcription (RT) primers, each having a 3′ poly(T) sequence, a cell specific barcode sequence, a unique molecular identifier (UMI) barcode, a next generation sequencing (NGS) region and ISPCR sequence;
(iii) generating a complementary strand for the reverse transcribed RNAs obtained in step (ii) using (1) template switching oligonucleotides (TSO) bound to ISPCR primers, and (2) at least one type of gene-specific primers bound to ISPCR primers;
(iv) amplifying the generated complementary strands using PCR; and
(v) tagmenting the amplified products with a transposase for fragmentation and insertion of transposon adapter sequence.

According to an additional aspect, the present invention provides a method for preparing a library of nucleic acids for sequence analysis of single-cell transcriptomes, comprising:
(i) providing a plurality of RNA populations of individual cells, wherein the RNA populations being separated;
(ii) reverse-transcribing the plurality of RNA populations using a plurality of reverse transcription (RT) primers, each having a 3′ poly(T) sequence, a cell specific barcode sequence, a unique molecular identifier barcode, a next generation sequencing (NGS) region and ISPCR sequence;
(iii) generating a complementary strand for the reverse transcribed RNAs obtained in step (ii) using at least one type of gene-specific primers bound to ISPCR primers;
(iv) amplifying the generated complementary strands using PCR; and
[symbol] agmenting the amplified products with a transposas [symbol] n and insertion of transposon adapter sequence.

According to some embodiments, the method comprises a step of pooling. According to some embodiments, the pooling is performed before the step of amplification. According to some embodiments, 5, 8, 10, 12, 20, 30, 40, or 50 of the RNA populations or more are pooled. According to some embodiments, more than 100, 200, 500, 1000, 5000 or 10000 of the RNA populations are pooled. According to other embodiments, the pooling is performed after the step of amplification.

According to some embodiments, the tagmentation is performed with a single type of transposon having a single, identical adapter sequence. According to some embodiments, tagmentation is performed using the Tn5 transposase. According to additional embodiments, the tagmentation is performed with different types of transposons.

According to some embodiments, the reverse transcription is performed using MMLV reverse transcriptase (MMLV RT).

According to some embodiments, the method further comprises a step (vi) of indexing to enable a second step of pooling of different plates or libraries.

According to some embodiments, the method comprises an additional step of pooling following the tagmentation step.

According to some embodiments, the reverse transcription primer and/or the PCR primer comprises an index sequence enabling the pooling of different plates or libraries.

According to some embodiments, the next generation sequencing (NGS) region comprises a P5 primer sequence, P7 primer sequence, an index sequence, Read 1 primer sequence and/or Read 2 primer sequence. According to some embodiments, the next generation sequencing region comprises a P5 primer sequence or P7 primer sequence. According to some embodiments, the next generation region comprises an index sequence. According to some embodiments, the next generation sequencing region comprises Read 1 or Read 2 primer sequence that is used during NGS sequencing.

According to some embodiments, the method comprises an additional step (vi) comprising the addition of a second next generation sequencing region. According to some embodiments, the method comprises an additional step (vi) of amplifying and selecting the desired products using primers containing NGS sequences, which are complementary to adapter sequences.

[symbol] rding to certain embodiments, the second next gener [symbol] egion comprising an index sequence. According to exemplary embodiments, the second next generation sequencing region comprising P5 or P7 primer sequences. According to some embodiments, the second next generation sequencing region comprises a Read 2 or Read 1 sequence.

According to some embodiments, the PCR amplification is performed with ISPCR primers.

According to some embodiments, the second next generation sequencing region is added by a PCR amplification step where the NGS region is part of the primer. According to some embodiments, the NGS region is annealed to the Tn5 adapter sequences. According to other embodiments, the second next generation sequencing region is added by a ligation reaction.

According to some embodiments, step (ii) and step (iii) are performed substantially simultaneously. According to certain embodiments, step (ii) and step (iii) are performed in a single reaction step. According to exemplary embodiments, the RNA populations are contacted with the RT primer, a reverse transcriptase, TSO, gene-specific primers, and dNTPs. According to these embodiments, step (ii) and step (iii) are performed in the same reaction mixture. According to other embodiments, the reaction buffer or the conditions are altered between steps (ii) and (iii).

According to some embodiments, the reverse-transcription step is performed on more than 5, 8, 10, 12, 15, 20, 30, 50, 100, 200, 500, 1000, or 5000 RNA populations. Each possibility represents a separate embodiment of the invention.

According to some embodiments, the UMIs have a length of between 4-12 nucleic acids. According to certain embodiments, the UMIs have a length of 4, 5, 6, 7, 8, 9, or 10 nucleic acids. Each possibility represents a separate embodiment of the invention.

According to some embodiments, the cell specific barcode length is between 6 and 12 nucleic acids. According to certain embodiments, the cell specific barcode length is 6, 7, 8, 9, 10, 11, 12, 13 or 14 nucleic acids. Each possibility represents a separate embodiment of the invention.

According to some embodiments, the step of generating a complementary strand is performed using a proofreading polymerase. According to additional embodiments, the amplification step is performed using a proofreading polymerase.

[symbol] rding to some embodiments, step (ii) is applied on a p [symbol] ments each has a single cell or cell lysate. According to some embodiments, the compartments comprise RNA inhibitors. According to some embodiments, the compartments present in a well plate. According to certain exemplary embodiments, the well plate is a 96-well plate. According to additional exemplary embodiments, the well plate is a 384-well plate.

According to some embodiments, the gene-specific primers are inserted into the well plate before adding the RNA population or a single cell. According to some embodiments, the gene-specific primers and the template switching oligonucleotides (TSO) are inserted into the well plate before adding the RNA population or a single cell.

According to some embodiments, the amplification step is a PCR reaction comprising more than 5, 10, 15, 20, 25, or 30 cycles. According to some embodiments, the amplification step is a PCR reaction comprising between 5 and 10 cycles, between 10 to 15 cycles, between 5 to 20 cycles, or more than 20 cycles. According to certain exemplary embodiments, the PCR reaction comprising between 15 and 25 cycles. According to additional exemplary embodiments, the PCR reaction comprises between 18 and 22 cycles.

According to some embodiments, the method further comprises a sequencing step. The sequencing method may be next generation sequencing (NGS) methods or any other sequencing method known in the art. According to some embodiments, the sequencing method is a next generation sequencing (NGS) method. According to certain embodiments, the next generation sequencing (NGS) method is based on the Illumina sequencing platform.

According to some embodiments, the cells are eukaryotic cells. According to some embodiments, the cells are animal cells. According to some embodiments, the cells are mammalian cells. According to certain embodiments, the cells are human cells.

According to some embodiments, the RNA populations comprise RNA populations of different tissues. According to certain embodiments, the RNA populations comprise RNA populations of cells from a patient and a corresponding healthy subject. According to certain embodiments, the pooling step comprises a separate pooling of different types of RNA populations.

According to some embodiments, the gene-specific primers are complementary to set of genes lowly expressed. According to some embodiments, the gene-specific primers are complementary to a gene of a family selected from the group consisting of chemokines, cytokines, immune checkpoint genes, signal transduction genes, transcription factors, and their corresponding receptors.

[symbol] rding to some embodiments, the gene-specific primers [symbol] y to a gene selected from the group consisting of CD4, CD8, CD3, FOXP3, T-bet, Eomes, Gata3, Rora, Rorc, Tcf-1, Bcl11b, RORgt, Ahr, Notch, Runx1, Tgfb1, Ifng, Ifngr1, Alox5, Irf4, Irf7, Ccl1, Ccl4, Ccl5, Ccl20, Ccr7, IcosL, Ccl3, I11, I12, I14, I15, I16, I17, I19, I110, I112b, I113, I116, I117, I125, I133, TSLP, Ltb, Lta, amphiregulin, I15ra, I123rb, IL17ra, I117rb, I127ra, Tigit, PD1, PDL1, ICOS, CTLA4, B7, CD28, CD112, CD155, Tlr1, Tlr2, Tlr3, Tlr4, Tlr5, Tlr6, Tlr7, Myd88, Stat1, and Stat3. Each possibility represents a separate embodiment of the invention.

According to some embodiments, the gene-specific primers are complementary to a sequence located between about 200-2500, 500-1000, 1000-2000, 1000-1500, or 1500-2500 bp upstream to the poly(a) sequence.

According to some embodiments the gene specific primers are between 18-22 base pairs in length. According to certain exemplary embodiments the gene specific primers are 20 base pairs in length.

According to some embodiments, the generation of a complementary strand for the reverse transcribed RNAs obtained in step (ii) uses 2, 3, 4, 5, or more different gene-specific primers bound to ISPCR primers. According to some embodiments, the different gene-specific primers are of the same gene.

According to some embodiments, the generation of a complementary strand for the reverse transcribed RNAs obtained in step (ii) uses 2 or more primers of different genes, each bound to an ISPCR primer.

According to some embodiments, the method comprises a step of processing tissue into a single cell suspension prior to step (i). According to certain embodiments, the method comprises a step of sorting the cells by FACS.

According to some embodiments, step (i) further comprises lysing the cells. According to some embodiments, the cells are lysed using a lysis reagent. According to certain embodiments, the lysis reagent is a detergent, a non-denaturing lytic detergent, a base, an acid, and/or an enzyme. According to some embodiments, the method further comprises a step of neutralizing the lysis reagent prior to any subsequent step. According to some embodiments, the cells are lysed using a hypotonic solution. According to other embodiments, the cells are lysed by a mechanical force. According to additional embodiments, the cells are lysed by high temperature.

According to some embodiments, the method further comprises a step of sequencing and analyzing the results.

[symbol] rding to an additional aspect, the present invention pro [symbol] paring a library of nucleic acids for sequence analysis of single-cell transcriptomes, comprising:
(i) a plurality of reverse transcription primers each having a 3′ poly(T) sequence, a cell barcode sequence, a unique molecular identifier barcode, a next generation sequencing (NGS) region and an ISPCR sequence; and
(ii) a plurality of gene-specific primers connected to an ISPCR sequence.

According to some embodiments, the kit comprises template switching oligos (TSO). According to certain embodiments, the TSO connected to an ISPCR primer.

According to some embodiments, the kit comprises a plurality of different gene-specific primers, each corresponds to a different region within the same gene. According to some embodiments, the kit comprises 2 or more primers of different genes, each connected to an ISPCR primer.

According to some embodiments, the kit comprises a Tn5 transposase.

According to some embodiments, the next generation sequencing region comprises a P5 primer sequence or P7 primer sequence. According to some embodiments, the next generation region comprises an index sequence. According to some embodiments, the next generation sequencing region comprises read 1 and/or read 2 primer sequence that is used during library amplification.

According to some embodiments, the kit comprises a reverse transcriptase, polymerase, reaction buffer, and/or dNTPs. According to some embodiments, the polymerase is a proofreading polymerase. According to additional embodiments, the polymerase is Taq polymerase.

Further embodiments and the full scope of applicability of the present invention will become apparent from the detailed description given hereinafter. However, it should be understood that the detailed description and specific examples, while indicating preferred embodiments of the invention, are given by way of illustration only, since various changes and modifications within the spirit and scope of the invention will become apparent to those skilled in the art from this detailed description.

### BRIEF DESCRIPTION OF THE FIGURES

FIG. 1 illustrates the WRAP-seq library preparation workflow.

[symbol] A-2B show comparative sequencing sensitivities of W [symbol] -seq. FIG. 2A—Number of detected genes (Y-axis) as a function of sequencing depth (X-axis). FIG. 2B—Number of detected unique molecular identifier (UMIs) as a function of sequencing depth. Pvalue <0.001 (Wilson's test).

FIG. 3 illustrates the TRAP-seq method. TRAP-seq is performed on the WRAP-seq platform, with the addition of gene-specific primer at the step of generating the complementary strand. The final library includes both target genes libraries and whole transcriptome libraries.

FIG. 4. Enrichment of CD4 expression analysis using the TRAP-seq method. qPCR of CD4 cDNA was performed on bulk RNA-seq of WRAP or TRAP (using a CD4 gene specific primer) libraries made for sorted CD4 cells, as indicated. Polyubiquitin-C (UBC) and CD45 were used as control.

### DETAILED DESCRIPTION OF EMBODIMENTS OF THE INVENTION

The present invention provides improved methods of transcriptomic analysis at the single-cell level. The methods described herein are rapid, accurate and cost-effective, and enable the analysis of many cells in parallel. In particular, the present invention combines the analysis of the whole transcriptome with even more accurate quantification and detection of specific, rare transcripts and/or genes of interest. The methods of the invention utilize the specific labeling of RNA populations of individual cells, and unique barcodes of RNAs that allows an early step of pooling that subsequently reduces costs and time in downstream processing steps. The methods of the invention enable a pooling step before downstream amplifications and utilize single types of transposons that reduce the loss of data.

The methods of the invention described the production of libraries for sequencing of RNA populations of individual cells. The library preparation workflow includes five steps; 1. reverse transcription, 2. generation of a second, complementary strand, 3. pooling, 4. amplification, 5. Tagmentation, and 6. 3′ product selection. The methods described herein incorporate cell barcodes for pooling libraries, and hence has the ability of processing different RNA population of individual cells together, reducing batch effects and increasing throughput. In addition, the libraries have UMIs to allow accurate transcript quantification.

According to an aspect, the present invention provides a method for preparing a library of nucleic acids for sequence analysis of single-cell transcriptomes, comprising:
[symbol] roviding a plurality of RNA populations of indi [symbol] RNA populations being separated;
(ii) reverse-transcribing the plurality of RNA populations using a plurality of reverse transcription (RT) primers, each having a 3′ poly(T) sequence, a cell barcode sequence, a unique molecular identifier barcode, a next generation sequencing (NGS) region and ISPCR sequence;
(iii) generating a complementary strand for the reverse transcribed RNAs obtained in step (ii) using (1) template switching oligonucleotides (TSO) bound to ISPCR primers, and (2) at least one type of gene-specific primers bound to ISPCR primers;
(iv) amplifying the generated complementary strands using PCR with ISPCR primers; and
(v) tagmenting the amplified product with a transposase for fragmentation and insertion of a transposon adapter sequence;
   thereby preparing a library of nucleic acids for sequence analysis of single-cell transcriptomes.

According to an additional aspect, the present invention provides a method for preparing a library of nucleic acids for sequence analysis of single-cell transcriptomes, comprising:
(i) providing a plurality of RNA populations of individual cells, the RNA populations being separated;
(ii) reverse-transcribing the plurality of RNA populations using a plurality of reverse transcription (RT) primers, each having a 3′ poly(T) sequence, a cell barcode sequence, a unique molecular identifier barcode, a next generation sequencing (NGS) region and ISPCR sequence;
(iii) generating a complementary strand for the reverse transcribed RNAs obtained in step (ii) using at least one type of gene-specific primers bound to a second ISPCR sequence;
(iv) amplifying the generated complementary strands using PCR with ISPCR primers; and
(v) tagmenting the amplified product with a transposase for fragmentation and insertion of a transposon adapter sequence;
   [symbol] ereby preparing a library of nucleic acids for sequenc [symbol] e-cell transcriptomes.

According to an additional aspect, the present invention provides a method for preparing a library of nucleic acids for sequence analysis of single-cell transcriptomes, comprising:
(i) providing a plurality of RNA populations of individual cells, the RNA populations being separated;
(ii) reverse-transcribing the plurality of RNA populations using a plurality of reverse transcription (RT) primers, each having a 3′ poly(T) sequence, a cell barcode sequence, a unique molecular identifier barcode, and ISPCR primer;
(iii) generating a complementary strand for the reverse transcribed RNAs obtained in step (ii) using at least one type of gene-specific primers bound to ISPCR primers;
(iv) amplifying the generated complementary strands using PCR with the ISPCR primer; and
(v) tagmenting the amplified product with a transposase for fragmentation and insertion of a transposon adapter sequence.

According to some embodiments, the method comprises a step of adding a next generation sequencing (NGS) region. According to some embodiments, the NGS region is added during an amplification step, the NGS region is part of the primer. According to other embodiments, the NGS region is added in a step of ligation following tagmentation.

According to additional embodiments, the method comprises a step of generating a complementary strand using a template switching oligonucleotide (TSO).

According to an additional aspect, the present invention provides a method for preparing a library of nucleic acids for sequence analysis of single-cell transcriptomes, comprising:
(i) providing a plurality of RNA populations of individual cells, the RNA populations being separated;
(ii) reverse-transcribing the plurality of RNA population using a plurality of reverse transcription (RT) primers, each having a 3′ poly(T) sequence, a cell barcode sequence, a unique molecular identifier barcode, and a next generation sequencing (NGS) region;
[symbol] enerating a complementary strand for the reverse tra [symbol] tained in step (ii) using (1) template switching oligonucleotides (TSO) bound to ISPCR primers, and (2) at least one type of gene-specific primers bound to ISPCR primers;
(iv) amplifying the generated complementary strands using PCR; and
(v) tagmenting the amplified product with a transposase for fragmentation and insertion of transposon adapter sequence.

According to an additional aspect, the present invention provides a method for preparing a library of nucleic acids for sequence analysis of single-cell transcriptomes, comprising:
(i) providing a plurality of RNA populations of individual cells, the RNA populations being separated;
(ii) reverse-transcribing the plurality of RNA population using a plurality of reverse transcription (RT) primers, each having a 3′ poly(T) sequence, a cell barcode sequence, and a next generation sequencing (NGS) region;
(iii) generating a complementary strand for the reverse transcribed RNAs obtained in step (ii) using (1) template switching oligonucleotides (TSO) bound to ISPCR primers, and (2) at least one type of gene-specific primers bound to ISPCR primers;
(iv) amplifying the generated complementary strands using PCR; and
(v) tagmenting the amplified product with a transposase for fragmentation and insertion of transposon adapter sequence.

According to some embodiments, the RT primer further comprises a unique molecular identifier barcode. According to certain embodiments, the RT primer further comprises ISPCR primer.

### Single Cell Preparation

Single-cell isolation is the first step for obtaining transcriptomic information from individual cells. Cells' isolation may be performed using any method known in the art. As used herein the term “isolation”, when used in the context of an isolated cell, refers to a specific target cell which has been artificially and purposefully removed from its natural environment and translocated to an environment where it can be further manipulated or [symbol] olated” cells, as indicated by this term, are present in [symbol] urified samples comprising a substantial percentage of said cells.

The term “RNA population” as used herein refers to complete RNA transcripts within an individual cell or extracted from individual cell. A plurality of RNA populations refers to the RNA transcripts of plurality of cells. The plurality of cells may be of the same or different tissues, from same or different individuals, and/or from cells that were under different conditions.

First, tissue is processed into single cell suspension and then, in some embodiments, the cells are sorted by FACS (allowing specific usage of markers) to capture hundreds or thousands of cells into 96- or 384-wells plates. The term “tissue” refers to any biological specimen obtained from any source such as a human, animal, or plant tissue. Examples of tissues include, without limitation, a biopsy sample, a cellular conglomerate, an organ fragment, whole blood, bone marrow, a fine needle aspirate, or any other solid, semi-solid, gelatinous, frozen or fixed three dimensional or two dimensional cellular matrix of biological origin. The processing of said tissue sample into a single cell suspension can be performed using a system that can utilize mechanical and enzymatic or chemical processes on a solid or liquid tissue sample and thus reduce said sample into single cells, nuclei, organelles, and biomolecules. In some embodiments, the tissue processing system performs affinity or other purifications to enrich or deplete cell types, organelles such as nuclei, mitochondria, ribosomes, or other organelles, or extracellular fluids.

A single cell suspension can be obtained using standard methods known in the art including, for example, enzymatically using trypsin or papain to digest proteins connecting cells in tissue samples or releasing adherent cells in culture, or mechanically separating cells in a sample. Single cells can be placed in any suitable reaction vessel in which single cells can be treated individually. For example, a 96-well plate, 384-well plate, or a plate with any number of wells such as 1000, 2000, 4000, 6000, 10000 or more. The multi-well plate can be part of a chip and/or device. The present invention is not limited by the number of wells in the multi-well plate. According to certain embodiments, the number of wells on the plate is from 80 to 200,000, 500 to 100,000 or 5000 to 10,000. According to other embodiments the plate comprises smaller chips, each of which includes 5,000 to 20,000 wells. For example, a square chip may include 125 by 125 nano-wells, with a diameter of 0.1 mm.

According to other embodiments, the sorted cells can be subjected into droplet-based sequencing using 3′ scRNA-seq of oil-droplet encapsulated cells achieved by microfluidic chamber. According to some embodiments, single cells can be isolated in [symbol] ome embodiments, encapsulating single cells in drop [symbol] sing a microfluidic device that comprises a droplet generator. For example, a population of single cells may be flowed through a channel of a microfluidic device, the microfluidic device including a droplet generator in fluid communication with the channel, under conditions sufficient to effect inertial ordering of the cells in the channel, thereby providing periodic injection of the cells into the droplet generator to encapsulate single cells in individual droplets. In some embodiments, the method of encapsulating single cells in droplets comprises the addition of an immiscible phase fluid, e.g., oil, to generate an emulsion of droplets each containing a single cell. Additional description of cell encapsulation using microfluidic droplet generators is found, e.g., in U.S. Patent Application Publication No. 20150232942.

In some embodiments, a droplet in which a single cell is encapsulated comprises a polymeric material. For example, suitable polymeric materials may include interpenetrating polymer networks (IPNs); a synthetic hydrogel; a semi-interpenetrating polymer network (sIPN); a thermoresponsive polymer; and the like. For example, in some embodiments, a suitable polymer comprises a co-polymer of polyacrylamide and poly(ethylene glycol) (PEG). In some embodiments, to suitable polymer comprises a co-polymer of polyacrylamide and PEG, and further comprises acrylic acid.

In some embodiments, a droplet in which a single cell is encapsulated may be a microgel droplet. In such embodiments, a microgel droplet may be a hydrogel droplet comprising a hydrogel polymer. Suitable hydrogel polymers may include, but are not limited to the following: acetic acid, glycolic acid, acrylic acid, 1-hydroxyethyl methacrylate (HEMA), ethyl methacrylate (EMA), propylene glycol methacrylate (PEMA), acrylamide (AAM), N-vinylpyrrolidone, methyl methacrylate (MMA), glycidyl methacrylate (GDMA), glycol methacrylate (GMA), ethylene glycol, fumaric acid, and the like. Some hydrogel polymers require the use of a cross linking agent. Common cross-linking agents include tetraethylene glycol dimethacrylate (TEGDMA) and N,N′-methylenebisacrylamide. The hydrogel droplets can be homopolymeric, or can comprise co-polymers of two or more of the aforementioned polymers. Exemplary hydrogel droplets include, but are not limited to, a copolymer of poly(ethylene oxide) (PEO) and poly(propylene oxide) (PPO); Pluronic® F-127 (a difunctional block copolymer of PEO and PPO of the nominal formula EO₁₀₀-PO₆₅-EO₁₀₀, where EO is ethylene oxide and PO is propylene oxide); poloxamer 407 (a tri-block copolymer consisting of a central block of poly(propylene glycol) flanked by two hydrophilic blocks of poly(ethylene glycol)); a poly(ethylene oxide)-poly(propylene oxide)-poly(ethylene oxide) co-polymer with a nominal molecular weight of 12,500 Daltons and a PEO: PPO ratio of 2:1); a poly(N-isopropylacrylamide)-base hydrogel (a PNIPAAm-based hydrogel); a PNIPAAm- [symbol] -polymer (PNIPAAm-co-AAc); poly(2-hydroxyethyl [symbol] (vinyl pyrrolidone); and the like.

According to some embodiments, the cells are isolated using Fluorescence activated cell sorting (FACS) or Flow cytometry. According to some embodiments, the cells are isolated using micropipetting or micromanipulation. According to additional embodiments, the cells are isolated using microscope-guided capillary pipettes, or by other standard means.

The cells are then lysed to further processing. According to some embodiments, the RNA is used directly from the lysed cells by placing the cells in a suitable buffer, optionally in the presence of a detergent (including but not limited to Tween-20, CHAPs and/or Triton X100), so as to lyse the cells. Reverse transcription reaction components may then be added directly to the lysate without further isolation to generate cDNA from the cellular RNA.

Synthesis of cDNA from mRNA in the methods described herein can be performed directly on cell lysates, such that a reaction mix for reverse transcription is added directly to cell lysates. Alternatively, mRNAs can be purified after their release from cells. This can help to reduce mitochondrial and ribosomal contamination. mRNA purification can be achieved by any method known in the art, for example, by binding the mRNA to a solid phase. Commonly used purification methods include magnetic or paramagnetic beads (e.g., of Dynabeads® BcMag®, and MagaCell®). Alternatively, specific contaminants, such as ribosomal RNA can be selectively removed using affinity purification.

Cellular/nuclear RNA serves as the RNA template to the subsequent reverse transcription and library preparation. According to some embodiments, the RNA template is mRNA. According to some embodiments, the RNA template is a low-abundance RNA. According to some embodiments, the RNA template is a disease-associated RNA. According to some embodiments, the RNA template is an oncogene RNA. The size of the RNA template may be about 100, 200, 300, 500, or 700 bp; or 1, 1.5, 2, 2.5, 3, 4, 5, 7, or 10 kb (i.e., kilo base pairs). The size of the RNA template may be between 100 bp and 10 kb, 150 bp and 500 bp, 200 bp and 500 bp, 100 bp and 1 kb, 100 bp and 5 kb, 300 bp and 10 kb, 500 bp and 1 kb, 200 bp and 10 kb, 300 bp and 10 kb, 500 bp and 10 kb, 700 bp and 10 kb, 1 kb and 10 kb, 1.5 kb and 10 kb, 2 kb and 10 kb, 3 kb and 10 kb, 4 kb and 10 kb, or 5 kb and 10 kb. Each possibility represents a separate embodiment of the invention.

According to some embodiments, the RNA template is isolated from a cell culture or a tissue sample. According to some embodiments, the tissue sample is a fresh tissue sample, a fine-needle aspiration (FNA) biopsy, a frozen tissue sample, a fresh frozen tissue sample, a biofluid tissue sample, a paraffin-embedded and fixed tissue sample, or a formalin-fixed [symbol] dded (FFPE) tissue sample. According to some embodi [symbol] ample is a solid tissue sample. According to additional embodiments, the tissue sample is a biofluid sample. Advantageously, in some embodiments, the methods described herein may be used to detect and analyze low-abundance RNA, e.g., RNA from a solid tissue sample or a biofluid sample. Exemplary biofluid samples useful for methods described herein include blood, serum, plasma, amniotic fluid, cerebrospinal fluid, interstitial fluid, lymph, saliva, fine needle aspiration, or urine.

Following isolation of single cells, mRNA can be released from the cells by lysing the cells. Lysis can be achieved by, for example, heating or freeze-thaw of the cells, or by the use of detergents or other chemical methods, or by a combination of methods. However, any suitable lysis method can be used. A mild lysis procedure can advantageously be used to prevent the release of nuclear chromatin, thereby avoiding genomic contamination of the cDNA library, and to minimize degradation of mRNA. For example, heating the cells at 72° C. for 3 minutes in the presence of triton x100 is sufficient to lyse the cells while resulting in no detectable genomic contamination from nuclear chromatin. Alternatively, cells can be heated to 65° C. for 10 minutes in water or 70° C. for 90 seconds in PCR buffer II (Applied Biosystems) supplemented with 0.5% NP-40; or lysis can be achieved with a protease such as Proteinase K or by the use of chaotropic salts such as guanidine isothiocyanate.

According to some embodiments, the RNA template for cDNA is in a complex RNA sample. In certain embodiments, a cellular RNA sample is used. In other embodiments, a total RNA sample is used. In certain embodiments, the RNA sample is obtained from a tissue sample. According to still further embodiments, the RNA sample is obtained from a cell culture.

General methods for RNA extraction are known in the art. RNA may be extracted from paraffin embedded tissues. RNA may be extracted from cultured cells and tissue samples using a commercial purification kit according to the manufacturer's instructions, e.g., using Qiagen RNeasy mini-columns, MasterPure™, Complete DNA Kit, EPICENTRE® RNA Purification Kit, and Ambion, Inc., Paraffin Block RNA Isolation Kit, Tel-Test RNA Stat-60. In certain embodiments, the extracted RNA is an RNA sample or an isolated RNA sample.

### Reverse Transcription

The methods of the invention comprise a step of reverse transcription using RT primers comprising poly dTs, cell barcode, UMI, NGS region and ISPCR.

[symbol] ethods described herein comprise the addition of [symbol] erated cDNA includes a handle comprising the cell barcode, UMI, NGS region and ISPCR.

The poly dT stretch is designed to prime the reverse transcriptase at the poly A tail of the mRNA molecules.

The cells' barcodes are a domain that uniquely identifies the sample source of the nucleic acid being sequenced to enable sample multiplexing by marking every molecule from a given sample (e.g. a single cell within a well) with a specific barcode or “tag”.

According to some embodiments, the cell barcode has a length of between 3-15 nucleic acids. According to some embodiments, the cell barcode has a length of between 4-14 nucleic acids. According to some embodiments, the cell barcode has a length of between 5-14 nucleic acids. According to some embodiments, the cell barcode has a length of between 4-13 nucleic acids. According to some embodiments, the cell barcode has a length of between 5-12 nucleic acids. According to some embodiments, the cell barcode has a length of between 6-12 nucleic acids. According to some embodiments, the cell barcode has a length of between 4-12 nucleic acids. According to some embodiments, the cell barcode has a length of between 4-10 nucleic acids. According to some embodiments, the cell barcode has a length of between 6-10 nucleic acids. According to certain embodiments, the cell barcode has a length of 4, 5, 6, 7, 8, 9, 10, 11, 12, 13, 14, or 15 nucleic acids. According to certain exemplary embodiments, the cell barcode has a length of 10 nucleic acids.

The unique molecular identifiers or UMIs are random sequences. A single UMI sequence marks a single transcript during the reverse transcription step before pooling and amplification. During the analysis, UMI duplications are omitted, thus reducing noise coming from cDNA amplification.

According to some embodiments, the UMI has a length of between 3-15 nucleic acids. According to some embodiments, the UMI has a length of between 4-14 nucleic acids. According to some embodiments, the UMI has a length of between 5-14 nucleic acids. According to some embodiments, the UMI has a length of between 4-13 nucleic acids. According to some embodiments, the UMI has a length of between 5-12 nucleic acids. According to some embodiments, the UMI has a length of between 6-12 nucleic acids. According to some embodiments, the UMI has a length of between 4-12 nucleic acids. According to some embodiments, the UMI has a length of between 4-10 nucleic acids. According to some embodiments, the UMI has a length of between 6-10 nucleic acids. According to certain embodiments, the UMI has a length of 4, 5, 6, 7, 8, 9, 10, 11, 12, 13, 14, [symbol] cids. According to certain exemplary embodiments, th [symbol] of 10 nucleic acids.

The NGS region is used herein as a general term for a short sequence suitable to be utilized later in high throughput sequencing methods as known in the art.

According to some embodiments, the NGS region comprises a sequencing platform adapter. A sequencing platform adapter domain may include one or more nucleic acid domains of any length and sequence suitable for the sequencing platform of interest. In certain aspects, the nucleic acid domains are from 4 to 100 nts in length. For example, the nucleic acid domains may be from 6 to 75 nts in length, from 10 to 50, or from 10 to 40 nts in length. According to certain embodiments, the sequencing platform adapter construct includes a nucleic acid domain that is from 4 to 10, from 9 to 15, from 16 to 22, from 23 to 29, or from 30 to 36 nucleotides in length.

According to some embodiments, the NGS region comprises a domain (e.g., a capture site) that specifically binds to a surface-attached sequencing platform oligonucleotide (e.g., the P5 or P7 oligonucleotides attached to the surface of a flow cell in an Illumina® sequencing system). According to some embodiments, the NGS region comprises a P5 or P7 illumina adapter.

According to additional embodiments, the NGS region comprises a sequencing primer binding domain (e.g., a domain to which the Read 1 or Read 2 primers of the Illumina platform may bind).

The ISPCR, located at the 5′ end of the reverse transcription primer, are primers used for amplification following reverse transcription. The term “ISPCR primer” as used herein can be any sequence that can be used for amplification and adding additional elements, such as NGS region as described herein. A non-limiting example for ISPCR primer sequence is AAGCAGTGGTATCAACGCAGAGT (SEQ ID NO: 1), however a person skilled in the art may design and use any other suitable primer/adaptor as known in the art.

### Template Switching

According to some embodiments, the reverse transcriptase may have terminal transferase activity, where the enzyme is capable of catalyzing template-independent addition of deoxyribonucleotides to the 3′ hydroxyl terminus of a DNA molecule. In certain aspects, when the reverse transcriptase reaches the 5′ end of a template RNA, it is capable of incorporating one or more additional nucleotides at the 3′ end of the nascent strand not [symbol] template. For example, the reverse transcriptase is ca [symbol] ing 1, 2, 3, 4, 5, 6, 7, 8, 9, 10 or more additional nucleotides at the 3′ end of the nascent DNA strand.

According to some embodiments, a reverse transcriptase having terminal transferase activity incorporates 10 or less, 5 or less (e.g., 3) additional nucleotides at the 3′ end of the nascent DNA strand. All of the nucleotides may be the same (e.g., creating a homonucleotide stretch at the 3′ end of the nascent strand) or at least one of the nucleotides may be different from the other(s). According to some embodiments, the terminal transferase activity results in the addition of a homonucleotide stretch of 2, 3, 4, 5, 6, 7, 8, 9, 10 or more of the same nucleotides (e.g., all dCTP, all dGTP, all dATP, or all dTTP). According to certain embodiments, the terminal transferase activity results in the addition of a homonucleotide stretch of 10 or less, such as 9, 8, 7, 6, 5, 4, 3, or 2 of the same nucleotides. Each possibility represents a separate embodiment of the invention.

According to certain exemplary embodiments, the reverse transcriptase is an MMLV reverse transcriptase (MMLV RT). MMLV RT incorporates additional nucleotides (predominantly dCTP, e.g., three dCTPs) at the 3′ end of the nascent DNA strand. These additional nucleotides are useful for enabling hybridization between the 3′ end of the template switch oligonucleotide and the 3′ end of the nascent DNA strand, e.g., to facilitate template switching by the polymerase from the template RNA to the template switch oligonucleotide. For example, when a homonucleotide stretch is added to the nascent cDNA strand, the template switch oligonucleotide may have a 3′ hybridization domain complementary to the homonucleotide stretch to enable hybridization between the 3′ end of the template switch oligonucleotide and the 3′ end of the nascent cDNA strand.

According to some embodiments, the method comprises a template switching of the cDNA to produce a complementary strand. This step includes the addition of a PCR handle end sequence at an end opposite from the first handle end sequence. Template-switching (also known as template-switching polymerase chain reaction (TS-PCR)) is a method of polymerase reaction that relies on the addition of a primer through the activity of murine leukemia virus reverse transcriptase (see, e.g., Petalidis L. et al. Nucleic Acids Research. 2003; 31 (22): e142).

The reaction mixture includes the template switch oligonucleotide at a concentration sufficient to permit template switching of the polymerase from the template RNA to the template switch oligonucleotide. For example, the template switch oligonucleotide may be added to the reaction mixture at a final concentration of from 0.005 to 500 μM, 0.1 to 100 μM, 0.5 to 0.2 μM, 0.1 to 10 μM, 0.5 to 5 μM, or 2 to 4 μM. According to certain exemplary [symbol] the template switch oligonucleotide may be added to [symbol] re at a final concentration of about 0.9 μM.

The template switch oligonucleotide includes a 3′ hybridization domain and a 5′ ISPCR primer. The 3′ hybridization domain may vary in length, and in some instances ranges from 2 to 10 nucleic acids in length. The sequence of the 3′ hybridization domain, i.e., template switch domain, may be any convenient sequence, e.g., an arbitrary sequence, a heterpolymeric sequence or homopolymeric sequence (such as GGG), or the like.

According to some embodiments, the template switching oligonucleotide and/or the reverse transcription primer contains a locked nucleic acid (LNA) (bridged nucleic acid (BNA)). A blocked oligo strategy to prevent secondary template switching may be used.

The reverse transcription step, generation of a complementary strand, and the amplification step are performed in a reaction mixture having a pH suitable for primer extension reaction, template-switching, and PCR. According to some embodiments, the pH of the reaction mixture is between 5.5 and 9.5, 6 and 9, 6 and 8, 6.5 and 8.5 or 6.5 and 7.5. According to some embodiments, the pH is between 7 and 7.5, or 7.2 and 7.4 According to some embodiments, the reaction mixture comprises a pH adjusting agent. According to some embodiments, the pH adjusting agent is selected from the group consisting of sodium hydroxide, hydrochloric acid, phosphoric acid buffer solution, and citric acid buffer solution. According to these exemplary embodiments, the pH of the reaction mixture can be adjusted to the desired range by adding an appropriate amount of the pH adjusting agent. According to some embodiments, the pH is adjusted between two or more steps of the method.

The conditions of the reaction, for example time or temperature, for the reverse transcription step, producing of a complementary strand, amplification step and tagmentation. may vary according to factors such as the particular enzyme employed, and the melting temperatures of the primers employed. According to some embodiments, the reverse transcriptase is MMLV reverse transcriptase. The cDNA synthesis is generally carried out at temperatures between 37° C. and 42° C. According to other embodiments, the reaction conditions are between 10° C. and 70° C., 15° C. and 65° C., 20° C. and 60° C., 25° C. and 55° C., 30° C. and 60° C., 30° C. and 55° C., 30° C. and 50° C., or 35° C. and 55° C. Each possibility represents a separate embodiment of the invention. According to some embodiments, the cDNA synthesis is carried out in 42° C. for 90 min, followed by 10 cycles of 50° C. for 2 min and 42° C. for 2 min, then heat inactivation at 70° C. for 15 min and then hold at 4° C. According to some embodiments, the cDNA synthesis is carried out in 50° C. for 90 min, then heat inactivation at 85° C. for 5 min and then hold at 4° C.

[symbol] ording to some embodiments, the methods described [symbol] ooling step, the pooling step can be performed after or before amplification of the complementary strands produced from the cDNA molecules. As such, in certain embodiments of the methods described herein, cells are obtained from a tissue of interest and a single-cell suspension is obtained. A single cell is placed in one well of a multi-well plate, or other suitable container, such as a microfluidic chamber or tube. According to some embodiments, the cells are lysed and reverse transcription reaction mix is added directly to the lysates without additional purification. It is also possible that the container vessel also contains reverse transcription reagents when the cells are lysed. This results in the synthesis of cDNA from cellular mRNA and incorporation of a source (e.g., cell) barcode tag into the cDNA, e.g., as described above. The tagged cDNA samples are pooled and amplified, and then sequenced to produce reads. According to certain embodiments, the samples are amplified and then pooled. The process further comprises a tagmentation step.

A “pool” as used herein refers to multiple polynucleotide samples (for instance, 48 samples, 96 samples, or more) derived from the same or different organisms, as may be multiplexed into a single high-throughput sequencing analysis. Each sample may be identified in the pool by a unique sample barcode. The polynucleotides refer to the cDNAs produced from the RNA population and the complementary strands that were generated from the cDNA molecules. A “nucleotide sequence” or a “polynucleotide sequence” refers to any polymer or oligomer of nucleotides such as cytosine (represented by the C letter in the sequence string), thymine (represented by the T letter in the sequence string), adenine (represented by the A letter in the sequence string), guanine (represented by the G letter in the sequence string) and uracil (represented by the U letter in the sequence string). It may be DNA or RNA, or a combination thereof. It may be found permanently or temporarily in a single-stranded or a double-stranded shape. Unless otherwise indicated, nucleic acids sequences are written left to right in 5′ to 3′ orientation.

As described herein the methods may include a pooling step where a cDNA product composition, e.g., made up of synthesized first strand cDNAs or synthesized double stranded cDNAs, is combined or pooled with the cDNA product compositions obtained from one or more additional cells. The number of different cDNA product compositions produced from different cells that are combined or pooled in such embodiments may vary, where the number ranges in some instances from 50, 200, 500, 1000, 5000, 10000, 50000, 100000 or more. Prior to or after pooling, the product cDNA composition(s) can be amplified, e.g., by polymerase chain reaction (PCR), such as described above.

[symbol] rding to some embodiments, cells are obtained from [symbol] and a single-cell suspension is obtained. A single cell is placed in one well of a multi-well plate or other suitable container. The cells are lysed and reverse transcription reaction mix is added directly to the lysates without additional purification. This results in the synthesis of cDNA from cellular mRNA and incorporation of a source barcode tag into the cDNA. The tagged cDNA samples are pooled, amplified, and then sequenced to produce reads. This allows identification of genes that are expressed in each single cell.

“Amplification” refers to a polynucleotide amplification reaction to produce multiple polynucleotide sequences replicated from one or more parent sequences. Amplification may be produced by various methods, for instance a polymerase chain reaction (PCR), a linear polymerase chain reaction, a nucleic acid sequence-based amplification, rolling circle amplification, and other methods.

### Tagmentation

Tagmentation refers to a modified transposition reaction, often used for library preparation, and involves a transposon cleaving and tagging double-stranded DNA with a transposon adapter sequence. Tagmentation methods are known in the art. According to some embodiments, the tagmentation is performed using Transposase-assisted tagmentation of RNA/DNA hybrid duplexes, as described, for example, in Lu et al. (eLife 2020; 9:e54919).

The term “tagmentation” or “tagmenting” as used herein refers to the process that utilize the Tn5 transposon system for the simultaneous fragmenting of the cDNA to a shorter length and tagging the DNA with an adapter.

According to some embodiments, the tagmentation utilizes transposon complexes having two different adapter sequences. According to preferred embodiments, the transposon system described herein utilizes identical adapters having the same sequence. Tagging with adapters having the same sequence maintains high yield of products.

According to some embodiments, the tagmentation is conducted by incubating the PCR amplification product with a transposome complex comprised of transposase and transposon DNA to provide a population of dsDNA molecules. According to some embodiments, Tn5 transposase, or an active fragment or variant thereof, is used. Tn5 transposase mediates the insertion of DNA associated with short 19 base pairs ends. In some embodiments, the inserted sequence comprises Read 1 or read 2, and the total DNA inserted length is 33 or 34 bp.

[symbol] wing tagmentation, the original 3′ of the mRNA (5′ of [symbol] A) is amplified using a partial P7 primer and a primer specific to the transposon added sequence. Other products of the transposon-based reaction are not amplified, either because they lack all the necessary primer sites for amplification or because of suppression PCR. NGS regions (e.g., P5 sequence of illumina, cluster generation and indexing sequences) are added during the library amplification PCR stage to generate a library ready for sequencing.

The methods of the invention disclose the production of libraries preparation for in depth sequencing followed by computational analysis. Acceptable methods for next generation sequencing (NGS), including polynucleotide adapters and hybridization blockers, are known in the art.

The commonly used NGS workflows implement the steps of library preparation, including an adapter addition or ligation, surface attachment, and in-situ amplification. Advantageously, the adapters suitable for NGS in some embodiments, are incorporated during the steps of reverse transcription and amplification. These procedures are more efficient than the addition of adapters using ligation from both sides.

“Sequencing” refers to reading a sequence of nucleotides out of a DNA library to produce a set of sequencing reads which can be processed by a bioinformatics computer in a bioinformatics workflow. High throughput sequencing (HTS) or next-generation-sequencing (NGS) refers to real time sequencing of multiple sequences in parallel, typically between 50 and a few thousand base pairs per sequence. Exemplary NGS technologies include those from Illumina, Ion Torrent Systems, Oxford Nanopore Technologies, Complete Genomics, Pacific Biosciences, BGI, and others. Depending on the actual technology, NGS sequencing may require sample preparation with sequencing adapters or primers to facilitate further sequencing steps, as well as amplification steps so that multiple instances of a single parent molecule are sequenced, for instance with PCR amplification prior to delivery to flow cell in the case of sequencing by synthesis. “Sequencing depth” or “sequencing coverage” or “depth of sequencing” refers to the number of times a genome has been sequenced.

The NGS protocol will vary depending on the particular NGS sequencing system employed. Detailed protocols for sequencing an NGS library, e.g., which may include further amplification (e.g., solid-phase amplification), sequencing the amplicons, and analyzing the sequencing data are available from the manufacturer of the NGS sequencing system employed.

[symbol] NGS libraries produced according to the methods of the [symbol] e may exhibit a desired complexity (e.g., high complexity). The “complexity” of a NGS library relates to the proportion of redundant sequencing reads (e.g., sharing identical start sites) obtained upon sequencing the library. Complexity is inversely related to the proportion of redundant sequencing reads. In a low complexity library, certain target sequences are over-represented, while other targets (e.g., mRNAs expressed at low levels) suffer from little or no coverage. In a high complexity library, the sequencing reads more closely track the known distribution of target nucleic acids in the starting nucleic acid sample, and will include coverage, e.g., for targets known to be present at relatively low levels in the starting sample (e.g., mRNAs expressed at low levels). According to certain embodiments, the complexity of a NGS library produced according to the methods of the present disclosure is such that sequencing reads are produced for 70% or more, 75% or more, 80% or more, 85% or more, 90% or more, 95% or more, 96% or more, 97% or more, 98% or more, or 99% or more of the different species of target nucleic acids (e.g., different species of mRNAs) in the starting nucleic acid sample (e.g., RNA sample). The complexity of a library may be determined by mapping the sequencing reads to a reference genome or transcriptome (e.g., for a particular cell type). Specific approaches for determining the complexity of sequencing libraries have been developed, including the approach described in Daley et al. (2013) Nature Methods 10(4):325-327.

According to other embodiments, the NGS adapters are added to the library in a separate step. According to some embodiments, the NGS workflows comprises steps of cDNA fragmentation, DNA end-repair, surface attachment, and in-situ amplification. Fragmentation can be done for instance by mechanical shearing, sonification, enzymatic fragmentation and other methods. After fragmentation, the DNA pieces may be end repaired to ensure that each molecule possesses blunt ends. To improve ligation efficiency, an adenine may be added to each of the 3′ blunt ends of the fragmented DNA, enabling DNA fragments to be ligated to adapters with complementary dT-overhangs. These methods result in a “DNA-adapter product” that is compatible with a next-generation sequencing workflow.

Next generation sequencers are still limited in the total number of reads that they can produce in a single experiment (i.e. in a given run). The lower the coverage, the fewer reads per sample for the analysis, and the higher the number of samples that can be multiplexed within a next generation sequencing run. “Aligning” or “alignment” or “aligner” refers to mapping and aligning base-by-base, in a bioinformatics workflow, the sequencing reads to a reference genome or transcriptome sequence, depending on the application. As known in bioinformatics practice, in some embodiments “alignment” methods as employed herein may [symbol] certain pre-processing steps to facilitate the mapping [symbol] reads and/or to remove irrelevant data from the reads, for instance by removing non-paired reads, and/or by trimming the adapter sequence as the end of the reads, and/or other read pre-processing filtering means.

Exemplary bioinformatics data representations with different coordinate systems (absolute or relative position indexing, 0-based or 1-based, etc.) include the BED format, the GTF format, the GFF format, the SAM format, the BAM format, the VCF format, the BCF format, the Wiggle format, the GenomicRanges format, the BLAST format, the GenBank/EMBL Feature Table format, and others. “Coverage” or “sequence read coverage” or “read coverage” refers to the number of sequencing reads that have been aligned to a genomic position or to a set of genomic positions.

The process of single cell RNA sequencing is known in the art, and there are numerous notable methods which differ from one another in at least one of the following aspects: (i) cell isolation; (ii) cell lysis; (iii) reverse transcription; (iv) amplification; (v) transcript coverage; (vi) strand specificity; and (vii) UMI (unique molecular identifiers or tags that can be applied for the detection and quantification of unique transcripts). Another main point of comparison between the different methods is the coverage of the produced RNA transcript, whether it is a full length or nearly full-length transcript, a transcript corresponding to only the 3′-end, or the 5′-end. Acceptable methods for the production of a full-length RNA transcript include, but are not limited to the following methods: Tang, Quartz-seq, SUPER-seq, Smart-seq, Smart-seq2, MATQ-seq. Methods for the production of a 3′-end include but are not limited to CEL-seq, CEL-seq2, MARS-seq, MARS-seq2, InDrop, Drop-seq, SPLIT-seq, Seq-Well, sci-RNA-seq, Quart-seq2, Chromium, Cytoseq, STRT-seq and STRT/C1. Methods for the production of a 5′-end include but are not limited to, Chromium and DroNUC-seq. Compared to 3′-end or 5′-end counting protocols, full-length scRNA-seq methods have incomparable advantages in isoform usage analysis, allelic expression detection, and RNA editing identification due to their improved transcript coverage.

Notably, droplet-based technologies (e.g., Drop-seq, InDrop and Chromium) can generally provide a lager throughput of cells and a lower sequencing cost per cell compared to whole-transcript scRNA-seq. Thus, droplet-based protocols are suitable for generating huge amounts of cells to identify the cell subpopulations of complex tissues or tumor samples. Several scRNA-seq technologies can capture both polyA+ and polyA−RNAs, such as SUPER-seq and MATQ-seq. These protocols are useful for sequencing long noncoding RNAs (IncRNAs) and circular RNAs (circRNAs). Compared to traditional bulk RNA-seq [symbol] scRNA-seq protocols suffer higher technical variations. [symbol] te the technical variances among different cells, spike-ins (such as External RNA Control Consortium (ERCC) controls) and UMIs have been widely used in corresponding scRNA-seq methods. The RNA spike-ins are RNA transcripts (with known sequences and quantity) that are applied to calibrate the measurements of RNA hybridization assays, such as RNA-Seq, and UMIs can theoretically enable the estimation of absolute molecular counts. Notably, ERCC and UMIs are not applicable to all scRNA-seq technologies due to the inherent protocol differences. Spike-ins are used in approaches like Smart-seq2 and SUPeR-seq but are not compatible with droplet-based methods, whereas UMIs are typically applied to 3′-end sequencing technologies (such as Drop-seq, InDrop and MARS-seq).

The mapping ratio of reads is an important indicator of the overall quality of scRNA-seq data. Since both scRNA-seq and bulk RNA-seq technologies generally sequence transcripts into reads to generate the raw data in BAM or fastq format, no differences exist between these two types of RNA-seq data in read alignment. The mapping tools originally developed for bulk RNA-seq are also applicable to scRNA-seq data. Numerous spliced alignment programs have been designed for mapping RNA-seq data. Generally, the read mapping algorithms mainly fall into two categories: spaced-seed indexing based and Burrows-Wheeler transform (BWT) based. Currently popular aligners like TopHat2, STAR and HISAT perform well in mapping speed and accuracy, and they can efficiently map billions of reads to the reference genome or transcriptome. STAR is a suffix-array based method and is faster than TopHat2, but it requires a huge memory size (28 gigabytes for human genome) for read mapping. Different mapping tools exhibit distinct strengths and weakness, where some programs are with a faster mapping speed but a lower accuracy in splice junction detection. HISAT is developed based on BWT and Ferragina-Manzini (FM) methods. For gene/transcript expression quantification, distinct approaches are needed, based on the range of transcript sequence captured by scRNA-seq. The data generated by whole-transcript scRNA-seq methods (such as Smart-seq2 and MATQ-seq) can be analyzed with the software developed for bulk RNA-seq to quantify gene/transcript expression. Two main approaches are available for transcriptome reconstruction: de novo assembly (does not need a reference genome) and reference-based or genome-guided assembly. De novo transcriptome assembly methods are primarily applied to the organisms that lack a reference genome, and are generally with a lower accuracy than that of genome-guided assembly. The popular genome-guided assembly tools including Cufflinks, RSEM and Stringtie have been broadly used in many scRNA-seq studies to get relative gene/transcript expression estimation in reads or fragments per kilobase per million mapped reads (RPKM or FPKM) or transcripts per million mapped [symbol] or the 3′-end scRNA-seq protocols (e.g., CEL-seq2, M [symbol] q, and InDrop), specific algorithms are required to calculate gene/transcript expression based on UMIs. SAVER (single-cell analysis via expression recovery) is an efficient UMI-based tool recently proposed for accurately estimating gene expression of single cells. In theory, UMI-based scRNA-seq can largely reduce the technical noise, which remarkably benefits the estimation of absolute transcript counts.

Currently, the Illumina platform is widely used (e.g., HiSeq4000, NextSeq500, NovaSeq 6000 or miSeq) for the sequencing step. The method of the invention comprises the addition of next generation regions suitable for in depth sequencing. It should be understood that these regions may be easily replaced or adjusted to any in depth sequencing machinery as required.

The nucleotide sequences of the reverse transcription primer suitable for sequencing on a sequencing platform may vary and/or change over time. Adapter sequences and other technical requirements are typically provided by the manufacturer of the sequencing platform. The sequence of any sequencing adapter domains of the template switch oligonucleotide, first strand cDNA primer, amplification primers, etc., may be designed to include all or a portion of one or more nucleic acid domains in a configuration that enables sequencing the nucleic acids on the platform of interest.

According to another aspect, the present invention provides a kit for preparing a library of nucleic acids for sequence analysis of single-cell transcriptomes, comprising:
(i) a plurality of reverse transcription primers each having a 3′ poly(T) sequence, a cell barcode sequence, a unique molecular identifier barcode, and a next generation sequencing (NGS) region and an ISPCR primer; and
(ii) a plurality of gene-specific primers connected to an ISPCR primer.

According to some embodiments, the reverse transcription primer comprises a sequence of ISPCR sequence at the 5′ end. According to certain embodiments, the gene-specific primers are bound to ISPCR primers.

The term “gene-specific primer” as used herein refers to a primer having a sequence corresponding a specific gene and that allows for the generation of a complementary strand for the reverse transcribed RNAs. The invention described herein includes the use of different primers of the same gene and/or different primers, each is of a different gene. Primers of different genes may be used for amplifying a plurality of different genes having low expression.

[symbol] rding to some embodiments, the kit comprises te [symbol] ligos. According to some embodiments, the template switching oligos are bound to ISPCR primers.

According to some embodiments, the kit further comprises a transposome comprising a transposase and a transposon nucleic acid comprising a transposon adapter sequence. According to some embodiments, the kit comprises a Tn5 transposase. According to some embodiments, the kit comprises a primer comprising a transposon adapter bound to a next generation sequencing region.

According to some embodiments, the next generation sequencing region comprises a P5 primer sequence or P7 primer sequence. According to some embodiments, the next generation region comprises an index sequence. According to some embodiments, the next generation sequencing region comprises read 1 or read 2 primer sequence that is used during library amplification.

According to some embodiments, the kit further comprises reagents for conducting a nucleic acid amplification assay.

According to some embodiments, the kit comprises a reverse transcriptase, proofreading polymerase, reaction buffer, dNTPs, and/or Taq polymerase.

According to some embodiments, the kit comprises instructional material for the use of the kit.

As used herein, the term “about” when combined with a value refers to ±10% of the reference value.

As used herein the singular forms “a”, “an”, and “the” include plural references unless the context clearly dictates otherwise. Thus, for example, reference to “a compound” includes a plurality of such compounds. It should be noted that the term “and” or the term “or” are generally employed in their sense including “and/or” unless the context clearly dictates otherwise.

Additional objects, advantages, and novel features of the present invention will become apparent to one ordinarily skilled in the art upon examination of the following examples, which are not intended to be limiting. Additionally, each of the various embodiments and aspects of the present invention as delineated hereinabove and as claimed in the claims section below finds experimental support in the following examples.

### EXAMPLES

### Example 1

Well-Based RNA Amplification and Pooling

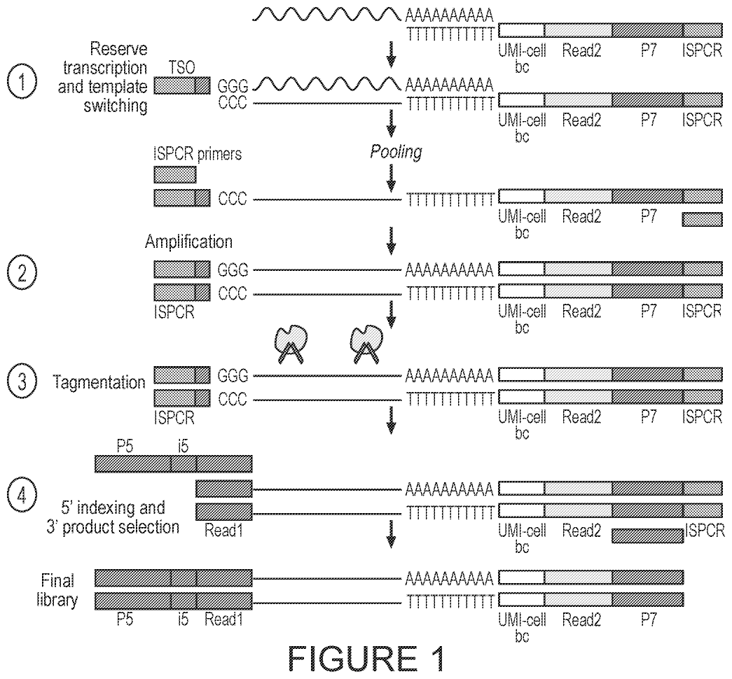

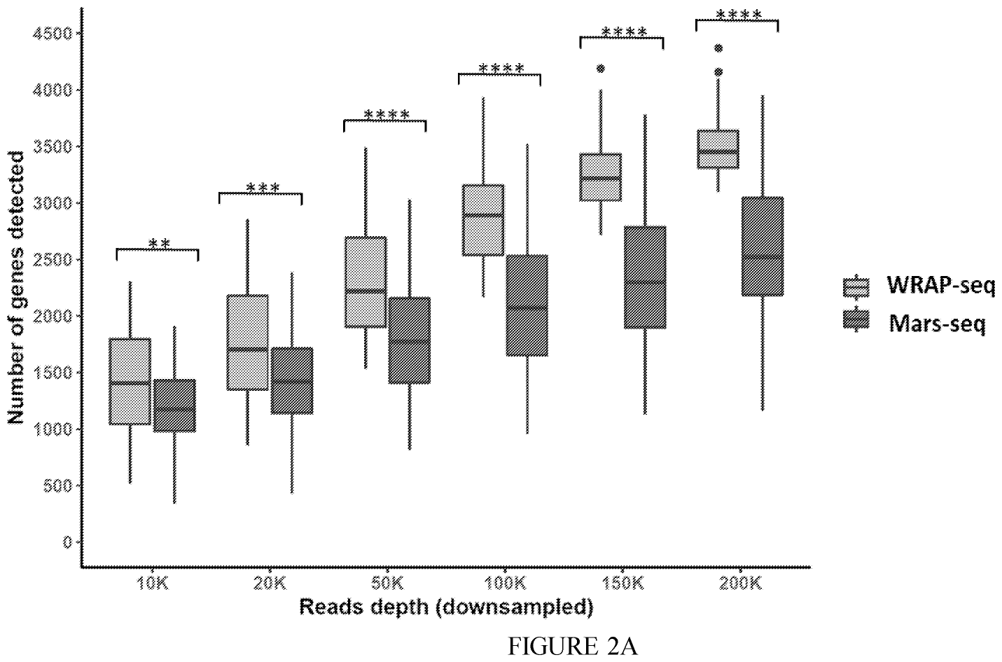

The WRAP-seq method that served as the basis to develop the TRAP method is schematically described in FIG. 1, and includes the following steps. Initially, the RNA is reversed transcribed using a dTs stretch connected to a cell barcode, UMI, NGS sequence, and ISPCR primer. Then, a complementary strand is synthesized using a template-switching oligo (TSO) for second-strand cDNA synthesis which is then amplified. The final steps include Tagmentation for fragmentation and tagging, and amplification with unique primers to select for fragments containing the 3′ end. A second NGS region, e.g., P5 and I5 are then added and the library is ready for sequencing.

To measure the sensitivity of the WRAP method, libraries were prepared from HEK293T cells and sequenced. The sensitivity was compared with Mars-seq, using a previously published Mars-seq dataset of HEK293T cells (Mereu, et al. Nature biotechnology 38.6 (2020): 747-755). The two datasets were analyzed together to avoid biases coming from analysis, and it was found that WRAP-seq was significantly more sensitive than Mars-seq (FIG. 2). The experiment included harvesting 293T cells (70% confluent) and sorted into 96-well plates. WRAP-seq protocol was then utilized to examine the sensitivity of the method. The analysis of the data was done by comparing the raw data of MARS-seq to the this of WRAP-seq.

### Example 2

### Single Cell Transcriptomic Using TRAP

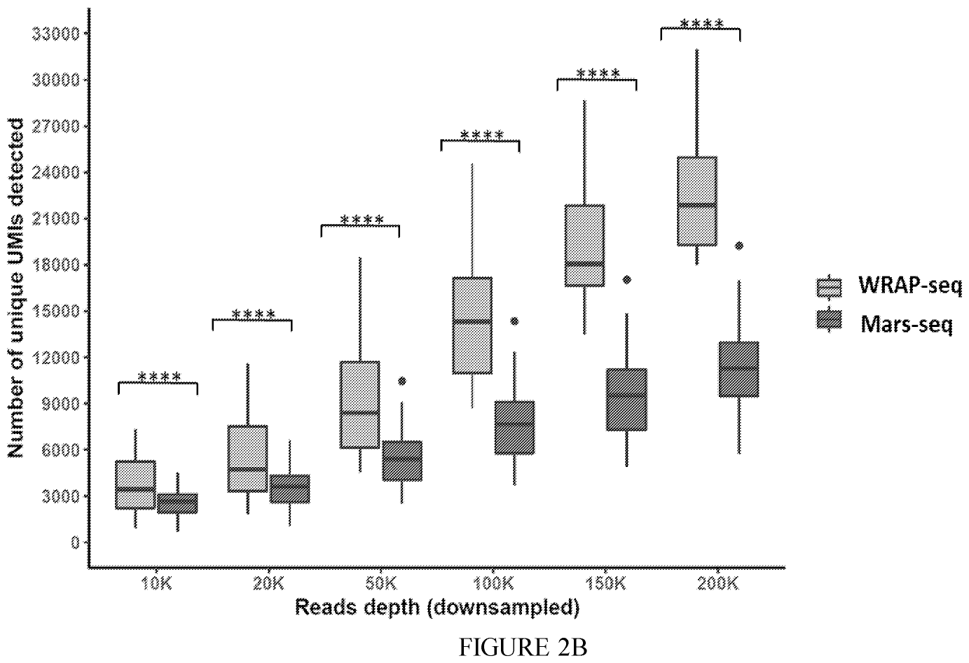

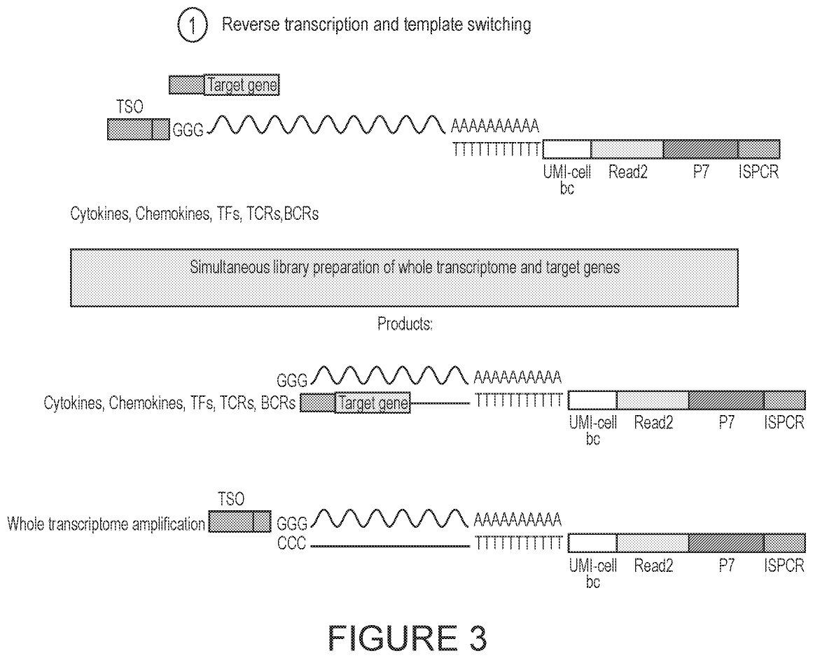

The WRAP-seq method described in Example 1 is used as a platform for targeted sequencing. Targeted sequencing is the specific detection of a panel of genes that are usually rarely detected using traditional scRNA-seq methods, due to low abundance or other limiting factors. Gene specific primers are added, together with/without Poly T primer and TSO to acquire whole transcriptome amplification (WTA) or specific-genes amplification, in order to capture needed or lowly expressed genes, such as TCR or BCR sequences, transcription factors and cytokines. Using Targeted Well-based RNA Amplification and Pooling sequencing (TRAP-seq; described in FIG. 3), sequencing resolution is increased, enabling identification of rare expressed genes together with whole transcriptome analysis. This novel [symbol] in depth analysis in a resolution that is currently un [symbol] isting methods.

In TRAP-seq, in addition to TSO for full-length transcriptome capture, an additional primer/s are introduced upstream to the 3′ end of a specific set of target genes. The targeting primer captures specific genes of interest, allowing selective enrichment of second-strand synthesis for target genes. The final library includes both target libraries and whole transcriptome amplification.

Preliminary steps undertaken prior to the performance of a TRAP-seq analysis include the preparation of barcoded plates, cell processing and sorting into single-cell plates.

Barcoded plates are prepared as follows: 96 or 384 unique 3′ Poly T mRNA capture primers (IDT) are consolidated with Ultra-pure water (UPW) to obtain a stock of 1 μM. Then, the stock is further diluted with lysis buffer (0.1% triton 100x, 0.5% RNase inhibitor) to a working concentration of 325 nM, in order to reach a 100 nM concentration during reverse transcription (RT) reaction.

Immune cell dissociation from intestinal lamina propria: Immune cells from the Lamina Propria are isolated enzymatically by incubating the small intestine with Liberase™ (100 μg/mL, Sigma) and DNaseI (10 μg/mL, Sigma) for 45 min at 37° C. Cells are then incubated with CD45 and EpCAM FACS-labeled antibodies for subsequent single-cell sorting.

HEK293T cell processing: Cells are seeded onto 10 ml plates, and cultures with DMEM media supplemented with 10% FBS, 1% Glutamine and 1% Pen-strep antibiotics (Termed 293T media). Media is changed every 2 days, and cells are split when confluence reaches 100%. For cell sorting, cells are dissociated using 1 ml Trypsin C EDTA solution, followed by 1 min incubation in 37° C. Then, trypsin is quenched using 9 ml 293T media, cells are collected into a conical tube and centrifuged at 300 g for 3 min. Then cells are re-suspended in 293T media and transferred into FACS tubes and are kept on ice until sorting.

After the cells have been processed, they undergo sorting into single-cell plates.

FACS cell sorting for single-cell plates: DAPI viability stain is added shortly before the sample is inserted to the FACS machine. Live single cells are gated by specific markers and selected for single cell sorting. A single cell is dispensed into each 96/384 plate well. Each plate contains 90 single cells, three empty wells for non-template control purposes, and three wells that contain two cells to account for doublets in the analysis. Sorted plates are [symbol] r 10 sec at 4° C., snap-freeze on dry ice, and stored [symbol] rary preparation.

After the preliminary steps have been completed, the TRAP-seq protocol can be performed on the single-cell plates containing the desired cells to be interrogated.

TRAP-seq protocol comprises the following: the target primer(s) are added during reverse transcription in order to increase the probability of target gene capture and reverse transcription.

The reverse transcription phase comprises the following steps: A sorted plate is placed on ice for 1-2 min until it thaws and then centrifuged at 800 g for 1 min at 4° C. Then, the plate is inserted into the PCR for 3 min at 72° C. and again centrifuged at 800 g for 30 seconds at 4° C. Next, the plate is placed on ice to cool for 2 min, followed by the addition of 4.5 μl of TRAP-RT mix (1 mM dNTP mix, RT buffer, 10 mM betaine, 10 mM MgCl₂, 100 nM TRAP primer, 1 μM TSO, 1 U/μl RNAse inhibitor, 2 U/μl RT enzyme) into each well with an additional centrifugation of at 800 g for 30 seconds at 4° C. Finally, the plate is inserted into the PCR for RT (90 min 50° C., 5 min 85° C.).

The amplification process comprises the following steps: the plate is centrifuged at 800 g for 1 minute, after which an amplification mix (0.2 μM ISPCR Primer, and PCR ready mix) is added to each well, followed by centrifugation at 800 g for 30 seconds at 4° C. The plate is inserted into the PCR for amplification, which includes the following steps: 98° for 3 minutes, 15-22 cycles of (98° C. for 15 seconds, 67° C. for 20 seconds, 72° C. for 6 minutes), final extension at 72° C. for 5 minutes and hold at 4° C. For single-cell usually perform 19 cycles for non-pooled amplification, or 21 cycles for pooled amplification.

Pooling: Pooling can be performed either before or after amplification. For pooling, all wells are combined and collected into an Eppendorf tube. Then, 10-30% of the product's volume is taken for further library preparation. The rest of the remaining library pool is stored at −20° C. If pooling occurs after amplification, an additional SPRI beads cleaning step is required.

Tagmentation: Using a purified Tn5 enzyme according to Picelli et al. Genome research 2014, that is loaded either with read1&2 or only with read1. Amplification and 3′ product selection—using a unique primer set that amplifies only tagmented products which contain the 3′ end.

TRAP-seq target primer panels: Each TRAP primer contains the ISPCR common sequence as a handle for amplification, and a sequence complementary to the target gene. [symbol] mer's length ranges around ˜50 bp (including the ISP [symbol] ding on the specific target gene.

Examples of gene-specific primers target:
1. Lymphocyte cell subsets identification
2. General Inflammatory response
3. Cytokines and chemokines
4. Immune checkpoints
5. Innate immunity
6. Signal transduction target genes
7. And more.

### Example 3

### Analyzing Specific Genes Abundance Using the TRAP-Seq Method

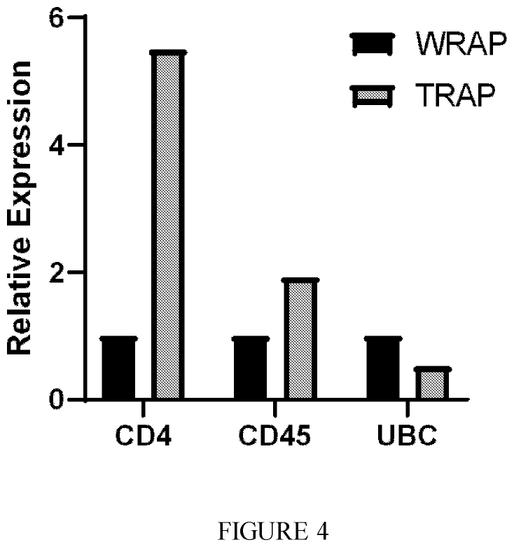

To examine the efficiency and sensitivity of the TRAP procedure, TRAP-seq library was compared with WRAP-seq library. The libraries were prepared from CD4+ T cells. The TRAP used CD4-specific primer. The analysis of CD4 expression was done via qPCR. As shown in FIG. 4, there was a ˜3-fold specific amplification of the CD4 target mRNA using the CD4-TRAP primer (TRAP CD4) compared with same CD4-expressing T cells that were processed using the WRAP-seq method (without the CD4 targeting (WRAP CD4)).

As a control, CD8 T cells that do not express CD4 underwent the same CD4-TRAP targeting or WRAP-seq library preparation, where non-specific products were not detected (not shown).

The same samples underwent qPCR analysis also for CD45 and UBC mRNAs expression, which were not specifically targeted and which are known to be expressed within all analyzed cell samples. Results show that both UBC and CD45 were expressed within all samples, and the amplification was not negatively affected in the TRAP method. The controls suggests that the CD4 amplification detected in the TRAP CD4 sample is not due to overall more material in the TRAP CD4 sample, but a specific CD4 amplification. The results confirm the specificity of the TRAP-seq protocol. Moreover, the detection of UBC and CD45 demonstrates that whole genome amplification occurs alongside target-specific amplification, and may be analyzed simultaneously.

Although the invention has been described in conjunction with specific embodiments thereof, it is evident that many alternatives, modifications, and variations will be apparent to those skilled in the art. Accordingly, it is intended to embrace all such alternatives, modifications and variations that fall within the spirit and broad scope of the appended claims.

1. A method for preparing a library of nucleic acids for sequence analysis of single-cell transcriptomes, comprising:
(i) providing a plurality of RNA populations of individual cells, the RNA populations being separated;
(ii) reverse-transcribing the plurality of RNA populations using a plurality of reverse transcription (RT) primers, each having a 3′ poly(T) sequence, a cell specific barcode sequence, a unique molecular identifier barcode, a next generation sequencing (NGS) region and ISPCR sequence;
(iii) generating a complementary strand for the reverse transcribed RNAs obtained in step (ii) using (1) template switching oligonucleotides (TSO) bound to ISPCR primers, and (2) at least one type of gene-specific primers bound to ISPCR primers;
(iv) amplifying the generated complementary strands using PCR; and
(v) tagmenting the amplified product with a transposase for fragmentation and insertion of transposon adapter sequence.

2. The method of claim 1, further comprising a step of pooling prior to step (iv).

3. The method of claim 2, wherein at least 48 RNA populations are pooled.

4. The method of claim 1, wherein the tagmentaion is performed with a single type of transposon having a single, identical adapter sequence.

5. The method of claim 1, wherein the reverse transcription is performed using MMLV reverse transcriptase (MMLV RT) or derivates thereof.

6. The method of claim 1, wherein the next generation sequencing (NGS) region comprises a P5 primer sequence, P7 primer sequence, an index sequence, Read 1 primer sequence and/or Read 2 primer sequence.

7. The method of claim 1, wherein the method comprises an additional step (vi) comprising the addition of a second next generation sequencing region.

8. The method of claim 1, wherein the PCR amplification is performed with ISPCR primers.

9. The method of claim 1, wherein the second next generation sequencing region is added by a PCR amplification step where the NGS region is part of the primer.

10. The method of claim 1, wherein step (ii) and step (iii) are performed substantially simultaneously or in a single reaction step.

11. The method of claim 10, wherein the RNA populations are contacted with the RT primer, a reverse transcriptase, TSO, gene-specific primers, and dNTPs.

12. The method of claim 1, wherein the reverse-transcription step is performed on more than 12 RNA populations.

13. The method of claim 1, wherein the UMIs have a length of between 4-12 nucleic acids.

14-15. (canceled)

16. The method of claim 1, wherein step (ii) is applied on a plurality of compartments each has a single cell or cell lysate.

17-18. (canceled)

19. The method of claim 1, wherein the method further comprising a step of producing an NGS library.

20-22. (canceled)

23. The method of claim 1, wherein the RNA populations comprise RNA populations of different tissues.

24. The method of claim 1, wherein the RNA populations comprise RNA populations of cells from a patient and a corresponding healthy subject.

25. The method of claim 1, wherein the pooling step comprises a separate pooling of different types of RNA populations.

26-29. (canceled)

30. A method for preparing a library of nucleic acids for sequence analysis of single-cell transcriptomes, comprising:
(i) providing a plurality of RNA populations of individual cells, the RNA populations being separated;
(ii) reverse-transcribing the plurality of RNA populations using a plurality of reverse transcription (RT) primers, each having a 3′ poly(T) sequence, a cell barcode sequence, a unique molecular identifier barcode, a next generation sequencing (NGS) region and ISPCR primer;
(iii) generating a complementary strand for the reverse transcribed RNAs obtained in step (ii) using at least one type of gene-specific primers bound to a ISPCR primers;
(iv) amplifying the generated complementary strands using PCR; and
(v) tagmenting the amplified product with a transposase for fragmentation and insertion of transposon adapter sequence.

31. A kit for preparing a library of nucleic acids for sequence analysis of single-cell transcriptomes, comprising:
(i) a plurality of reverse transcription primers each having a 3′ poly(T) sequence, a cell barcode sequence, a unique molecular identifier barcode, a next generation sequencing (NGS) region and an ISPCR primer; and
(ii) a plurality of gene-specific primers connected to an ISPCR primer.

32-35. (canceled)
